(19) United States
(12) Patent Application Publication
(10) Pub. No.: US 2010/0158841 A1
(43) Pub. Date: Jun. 24, 2010

(54) DEODORANT COMPOSITIONS

(75) Inventor: David Allen BREWSTER, Milford, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc., d/b/a UNILEVER, Englewood Cliffs, NJ (US)

(21) Appl. No.: 12/342,906

(22) Filed: Dec. 23, 2008

(51) Int. Cl. A61K 8/20; A61Q 15/00

(52) U.S. Cl. 424/65

(57) ABSTRACT

A gelled composition is provided which comprises:
(a) from 10 to 75% by weight water,
(b) gelling agent comprising an alkali metal salt of a C₁₂ to C₂₄ fatty acid and, optionally, a co-gellant,
(c) particles in the form of a synthetic oxide support on which is deposited a silver compound, and
(d) optionally, one or more emollients,
   wherein the gelled composition is in the form of a clear deodorant stick.

### BACKGROUND OF THE INVENTION

This invention relates to deodorant compositions in the form of gelled sticks for application to the human axillae, in particular, the underarms, to reduce malodor.

Deodorant compositions in stick form are often characterized on the basis of their being opaque or clear products. Opaque products commonly employ structurant that comprises a blend of waxy materials of different melting point, whereas, clear products are commonly structured by a gelling agent that, in the presence of other composition components, results in translucent or transparent products. As used herein “gelled sticks” or “gel sticks” are used interchangeably to refer to products wherein the gelling agent, together with water and other solvents present in such products, forms a gel network structure.

Compared to products structured by one or more waxy materials, gelled sticks, particularly sticks gelled by fatty acids or esters thereof, typically exhibit a relatively sharp transition from the liquid to solid phase. Wax sticks are commonly formulated as anhydrous or low water content compositions using a blend of waxy materials having different melting points. As a result, in wax sticks, the transition from the liquid to solid state generally does not take place at single defined temperature. In the liquid state, wax sticks are generally of higher viscosity than gelled sticks over a broader temperature range.

Owing to their relatively rapid solidification, obtaining gelled sticks of uniform composition can be difficult when incorporating additives that are relatively insoluble therein, particularly when those additives have a density significantly different from that of the gelled stick in its liquid state. When the composition of interest has relatively low levels of insoluble additives, failure to obtain uniform distribution of such additives is especially problematic.

Among the additives that can be difficult to uniformly incorporate into gelled sticks are metallic silver and silver salts. Given their limited water solubility and density (silver chloride, for example, has a density of 5.56 g/cc³ and a solubility in water of 52×10⁻⁶ g/100 g at 50° C.), silver salts tend to settle toward the bottom of gelled sticks, potentially giving rise to an uneven distribution of silver salt in the gelled products, and/or the requiring the use of specialized manufacturing techniques to counter the settling effect of the salts. Uneven distribution of silver salt may also give rise to undesirable coloring issues when the gel sticks are formulated for use as “clear” products.

Silver halides are among the deodorant active materials listed in U.S. Pat. No. 6,468,511, which discloses anhydrous cosmetic compositions comprising (a) 15-33% of an external phase which is made with at least one selected naphthalate organic ester; a volatile silicone based emulsifier; and a volatile silicone; and (b) 85-67% of an internal phase which is made with an active ingredient, in a glycol solution. The patent discloses that its oil/glycol emulsions can be used to form gel, soft solid or roll-on products such as antiperspirants and deodorants.

Relative to the number of gelled sticks that are commercially available, few contain silver or silver salts. Gelled sticks that contain silver or silver salts include, for example, a deodorant stick marketed by Herban Cowboy as ORGANIC GROOMING® Dusk Deodorant and a deodorant stick marketed by Herbal Clear as HERBAL CLEAR® Outdoor Whole Body Deodorant & Protection Stick. The ORGANIC GROOMING® Dusk Deodorant lists the following as ingredients: propylene glycol, Aloe barbadensis (aloe vera) leaf juice, silver, sodium stearate, fragrance, Rosmarinus officinalis (rosemary) leaf extract, Salvia officinalis (sage) leaf extract, Cucumis sativus (cucumber) fruit extract, Carum petroselinum (parsley) extract, and sodium bicarbonate. The HERBAL CLEAR® Outdoor Whole Body Deodorant & Protection Stick lists the following as ingredients: propylene glycol, water, stearic acid, steareth 20, silver citrate, Eucalyptus citriodora oil, Usnea barbata (lichen) extract, sodium chloride, Lavendula angustifolia (lavender) flower extract, Thymus vulgaris (thyme) extract, Juniperus virginiana (cedarwood) extract, Citrus medica limonium (lemon) fruit extract, Ferula assa foetida root extract, Eucalyptus globulous leaf oil, Lavendula angustifolia (lavender) oil, Citrus auranitium bergamia (bergamot) oil, Zingiber officinal (ginger) root oil, sodium hydroxide, cetyl alcohol, Vetikol, citric acid, and triethanolamine.

U.S. Pat. No. 6,444,726, discloses biocidal compositions comprising a sparingly soluble silver compound deposited on a synthetic oxidic support at a loading of 1-75% by weight of the support. Preferred silver compounds are silver chloride, with the patent disclosing that other silver compounds such as silver iodide, phosphate, hydroxide, carbonate, bromide, acetate, citrate, lactate, salicylate and stearate, as well as mixtures thereof, may also be used therein. The patent discloses that preferred supports are titania and other stable oxidic support comprising an oxide, hydroxide or oxy-anion species such as phosphate or sulfate. The patent further discloses that the biocidal component formed by depositing the silver compound on a synthetic oxide support can be used to form a concentrate composition, for example, a water-in oil or oil-in water type emulsion that, in turn, can be dispensed into a final composition. Among the compositions identified by the patent as compositions into which its biocidal component can be dispensed are: “leave on” and “rinse off” type cosmetics which can be subject to undesired bacterial growth, shampoos, sun-screens, water-based adhesives and paints, and detergent systems or liquid soaps.

Biocidal concentrates containing a synthetic oxidic support on which is deposited silver chloride are available commercially from Clariant AG under the trademark JM ACTICARE®. The use of JM ACTICARE® as an antimicrobial additive in a variety of cosmetic compositions has been disclosed. US 2003/0232091 discloses the use of JM ACTICARE® in various cosmetic compositions that include stabilized retinol. US 2005/0025729 discloses lipstick compositions that comprise a lipophilic phase in a concentration of 20 to 70% by weight, b) an aqueous phase in a concentration of 20 to 40% by weight, c) at least one Pickering pigment selected from the group consisting of titanium dioxide having a particle diameter of 10 to 100 nm and iron oxide having a particle diameter of 100 to 800 nm in an amount as therein more particularly described, d) at least one coated titanium dioxide pigment having a particle diameter of 100 to 2000 nm in an amount as therein more particularly described and e) further colored pigments in an amount of from 0.1 to 20% by weight; JM ACTICARE® is among the materials listed as suitable preservatives for use in the compositions therein described.

WO 01/01950 discloses pre-formed gel sheets comprising (a) less than 10% of a polysaccaharide mixture consisting of (i) a red seaweed polysaccharide, (ii) a mannose containing polysaccharide and (iii) a fermentation polysaccharide and (b) from about 30% to about 99.5% water; listed among the anti-microbial and anti-fungal actives suitable for used in the gel sheets is JM ACTICARE®.

US 2006/0115440 discloses the use of silver dihydrogen citrate-containing antimicrobial compositions, the silver dihydrogen citrate being generated as a stock solution prepared by applying electric potential to positive and negative electrodes, each consisting of 999 fine silver, which have been immersed in an aqueous solution of citric acid. At paragraph [0015] the application lists liquids, semi-solids, pastes, gels, bars, tablets, sprays, foams, powders and granules as suitable product forms for the compositions disclosed therein. The patent allows for the presence of additional antimicrobial agents and includes “silver compounds such as JM ActiCare” in the listing of additional antimicrobial agents that spans pages 5 and 6 of the application.

Achieving a uniform dispersion of a silver halide-containing antimicrobial agent in a gelled deodorant stick that solidifies from a fluid of relatively thin, near-water-like consistency to a clear solid at a relatively rapid rate is desired. More particularly, achieving a uniform dispersion of a silver halide -containing antimicrobial agent in deodorant composition in the form of a clear stick containing at least 10% by weight water and gelled with an alkali metal salt of a saturated fatty acid is desired.

### SUMMARY OF THE INVENTION

This invention relates to a gelled composition which comprises:
(a) from 10 to 75% by weight water,
(b) gelling agent comprising an alkali metal salt of a C₁₂ to C₂₄ fatty acid preferably a C₁₆ to C₂₂ fatty acid and, optionally, a co-gellant,
(c) particles in the form of a synthetic oxide support on which is deposited a silver compound, preferably a silver halide, and
(d) optionally, one or more emollients,
   wherein the gelled composition is in the form of a clear deodorant stick. Desirably the gelled composition is contained in a dispenser that includes instructions to apply the composition to the underarm.

### DETAILED DESCRIPTION OF THE INVENTION

As used herein all parts and percentages are by weight unless otherwise noted and are based on the entire weight of the composition unless otherwise noted. Except in the operating and comparative examples, or where otherwise explicitly indicated, all numbers in this description indicating amounts of material ought to be understood as modified by the word “about”. As used herein “essentially free of antiperspirant salts containing aluminum and/or zirconium” means that the total level of any such salts is too low to noticeably affect the clarity of the composition, which amount is typically an amount of 0.1% by weight of the composition or less. It is further noted that the use of “clear” in reference to the gelled compositions of the subject invention means that the composition is “transparent” or “translucent”. The term “transparent” as used in this specification is intended to connote its usual dictionary definition. Thus, a transparent cosmetic stick, like glass, allows for ready viewing of objects behind it. By contrast, a translucent cosmetic stick, although allowing light to pass through the stick, causes the light to be scattered so that objects behind the translucent stick are less clearly identified.

Within the context of this invention, a cosmetic composition is deemed to be “clear” if the transmittance of light of any wavelength in the range of 400 to 900 nm through a sample 1 cm thick is greater than 35%. In the practice of this invention, gelled sticks wherein the transmittance of light of any wavelength in the range of 400 to 900 nm, through a sample 1 cm thick, is greater than 50% are of particular interest, with sticks wherein such transmittance of light is greater than 60% being especially desirable. Light transmittance can be easily measured by placing a stick sample of the required thickness into the light beam path of a UV-VIS spectrophotometer such as a Baush & Lomb Spectrophotometer.

References to the compositions being “essentially free of waxy materials” means that the total amount of any waxes present does not noticeably effect solidification of the subject compositions compared to otherwise identical compositions in which such waxes are absent. Where compositions of the subject invention described as “including” or “comprising” specific compositions or materials, narrower embodiments where the compositions can “consist essentially of” or “consist of” the recited components or materials are also contemplated.

All of the ingredients used to prepare compositions of the invention are known or can be prepared according to known methods. The compositions of the invention can be prepared by known methods or by methods analogous to known methods.

### Water

The water that is used in the subject compositions is desirably de-ionized and, if desired, may be distilled. Deionization removes impurities which can interfere with gellation and/or form precipitates that can affect clarity. Desirably, water is present in the gelled compositions of this invention in an amount of at from 10 to 75% by weight, more particularly, from 15 to 40% by weight, and, in at least one embodiment of interest from 20 to 35% by weight. In at least one embodiment of interest wherein the composition includes from 40 to 65% by weight of emollient, water is desirably present in an amount of from 15 to 35% by weight.

If desired, a portion of the water may be replaced by one or more cosmetically acceptable organic solvents that are liquid at 0° C., miscible with water, and that do not undesirably effect gel formation and sensory properties of the resultant composition may be used herein. Ethanol is among the organic solvents that may be so employed. Organic solvent may be desirable as an aid in “lifting” fragrance.

### Gelling Agents

The gelling agent employed herein comprises an alkali metal salt of a fatty acid (herein also referred to as a “soap”), preferably a C₁₂ to C₂₄ fatty acid, more preferably a C₁₆ to C₂₂ fatty acid, with sodium and potassium salts being among the preferred alkali metal salts. Included among the fatty acids from which such salts are derived, include, for example, coconut oil, beef tallow, lanolin, fish oil, beeswax, palm oil, peanut oil, olive oil, cottonseed oil, soybean oil, corn oil, rapeseed, rosin acids and greases. In at least one embodiment of interest the alkali metal salts of fatty acids are selected from sodium stearate, potassium stearate, potassium palmitate, sodium myristate, and mixtures thereof.

The gelling agent may be added in salt form, or the salt may be generated in situ, such as, for example, by the reaction of the corresponding fatty acid and, for example, an alkali metal hydroxide. The gelling agent associates with water to form a gel network that provides structure to the composition. As fatty acids can potentially contribute to the stick having a hazy appearance, it is often preferable to add the salt form of the fatty acid, rather than generating the alkali metal salt of the fatty acid in situ. Further, given that alkali metal salts fatty acid salts can break down to their fatty acids, to shift the equilibrium between the alkali metal salts and their corresponding fatty acids and alkali metal hydroxide toward the salt form of the fatty acid, it is often desirable for the composition to be formulated to a pH of from about 8 to about 10.5, preferably from about 9 to about 10.

In addition to the alkali metal salt of a fatty acid, the gelling agent may further comprise one or more co-gellants. The use of co-gellant can provide a clarity stabilizing effect upon the composition. By replacing a portion of the fatty acid salt with a co-gellant, formulators may lessen the potential for fatty acid formation and, further, may provide compositions that are milder to the skin.

Included among the co-gellants suitable for use in the subject composition are polyethylene oxide-polypropylene oxide block copolymers of the general structure:

R_{f}[(C₂H₄O)ₐ(C₃H₆O)_{b}(C₂H₄O)_{c}(C₃H₆O)_{d}]ₑ[H]_{g} (I)

wherein:

R is independently selected from hydrogen, hydroxyl, C₁₀-C₂₂ fatty alkoxide, and ethylene diamine units;
a, b, c and d are independently selected integers ranging from 0 to 200 with the proviso that the sum of a, b, c and d is at least about 50;
e is an integer from 1 to 4;
f is an integer from 0 to 1; and
g is an integer from 0 to 4.

Included among such co-gellants are poly(ethylene oxide)(propylene oxide) (ethylene oxide) block copolymers commonly known as poloxamers. Typical of this substance are a series of products from BASF Corporation sold under the Pluronic® trademark. In at least one embodiment of interest, copolymers of this type will have an average molecular weight ranging from about 5000 to about 50,000, preferably between about 6,000 and 15,000. Melt/pour points of these materials should be at least 30° C., and optimally are at least 55° C. Illustrative commercially available poloxamers are Pluronic® F 127 block copolymer copolymers and Pluronic® F 108 block copolymers.

In the Formula I block copolymers, when f is 1 and R is an ethylene diamine unit, the general structure defines a tetra-functional copolymer derived from the sequential addition of propylene oxide and ethylene oxide to ethylene diamine and that may be described in terms of structures (II) and (III) as follows:

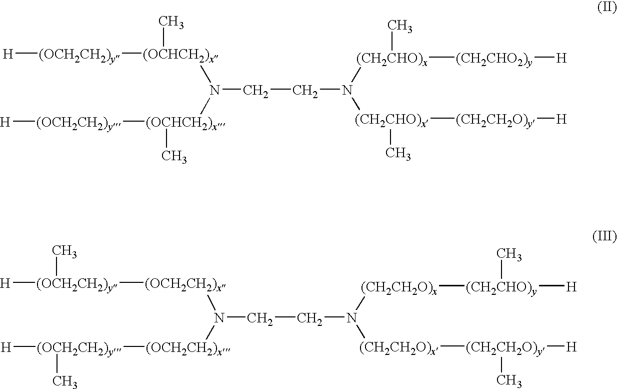

wherein X, X′, X″, X′″, Y, Y′, Y″, and Y′″ are integers such that the average molecular weight of the copolymer ranges from about 1,500 to about 100,000. Preferably, the average molecular weight should range from about 5,000 up to about 50,000, optimally between about 15,000 to about 30,000. In at least one embodiment of interest, these tetra-functional poloxamines are characterized as having a hydrophile-lipophile balance (HLB) of at least 12, preferably at least 18. HLB is an indicator of the relative attraction of a material for oil or water. Materials that are more oil soluble have lower HLB values, while materials that are more water soluble have higher HLB values. A method for determining HLB value is described by J. T. Davies in the Proceedings of the International Congress of Surface Activity (1957), pp. 426-438, which method is based on a material's molecular structure. Desirably, the poloxamines have a melt/pour point of at least 30° C., preferably greater than 40° C., and in at least one embodiment of interest, greater than 50° C. Suitable poloxamines are commercially available from BASF Corporation under the trademark, Tetronic®. Especially suitable are the Tetronic® 1107, Tetronic® 1307 and Tetronic 1508® block copolymers.

Desirably, the total amount of gelling agent is present in the subject composition in an amount sufficient to provide the composition with a self-supporting structure, however, the selection and amount of gelling agent should not undesirably detract from from the clarity desired in the ultimate product In the practice of this invention, total amounts of gelling agent of from 1 to 25% by weight of the composition, preferably from 3 to 20% by weight of the composition, more preferably 3 to 15% by weight of the composition are of particular interest. Ranges of preference depend, in part, on the choice and relative amounts of the gelling agent component(s), as well as the hardness and clarity desired in the gelled stick. In one embodiment of interest, when co-gellant is present, the total amount of the alkali metal salt of a fatty acid is desirably from 2 to 15% by weight of the composition, with amounts of from 3 to 10% by weight of the composition being of particular interest. When co-gellant is absent, the level of alkali metal salt of a fatty acid typically employed is from 5 to 25% by weight of the composition, more particularly from 5 to 20% by weight of the composition, and, in at least one embodiment, from 5 to 15% by weight of the composition. Desirably, the Formula I block copolymer is present in the subject compositions in an amount up to 6% by weight, preferably from 0.5 to about 5% by weight of the composition. In one embodiment of particular interest, the compositions of interest contain from 5 to 10% by weight of alkali metal salt of fatty acid and from 1 to 4% by weight of Formula I block copolymer.

Waxes and other organic or inorganic opacifying materials that, if present, otherwise detract from the clarity desired in the gel stick, should be kept to a level that does not undesirably interfere with stick clarity. Desirably, the total amount of such opacifying materials, exclusive of the silver halide-containing particles, should not exceed 1% by weight of the composition; preferably such opacifying materials should be absent or not exceed 0.5% by weight of the composition.

### Clarifying Agent

Optionally, the compositions of this invention may further comprise a clarifying agent for maintaining the clarity of the gelled composition. This agent is preferably a basic amine selected from amino alkanols having from 2 to 6 hydroxyl groups. These alkanols may have anywhere from 3 to 5 carbon atoms and have molecular weights less than 1,000. Particularly effective are the propanol amines. Ilustrative of this category is tetra(hydroxypropyl)diamine, available from BASF Corporation under the trademark, QUADROL®. Another preferred amino alkanol is 2-amino-2-methylpropan-1-ol, available from the Angus Chemical Company under the trademark, AMP®, Also suitable are 2-amino-2-ethyl-1,3-propanediol, available from the Angus Chemical Company under the trademark, AMPD® and 2-amino-2-hydroxymethyl-1,3-propanediol (generically referred to as tromethamine), available from the Siga Chemical company.

The basic amine may be present in amounts up to 2% by weight of the composition, more particularly, from 0.05 to 1% by weight of the composition. In an embodiment of particular interest, the basic amine is present in the compositions of this invention in an amount of from about 0.1 to about 0.6% by weight of the composition.

### Emollients

The compositions of the subject invention may further comprise one or more cosmetically acceptable emollients that are liquid at 20° C., such as, for example, cosmetically acceptable polyhydric alcohols containing from 2 to 6, preferably from 2 to 3, hydroxyl groups. Examples of such polyhydric alcohol emollients, include, for example, propylene glycol, dipropylene glycol, butylene glycol, glycerin, sorbitol, polyethylene glycol (for example, PEG 8, PEG 200 and PEG 400), and the like, and mixtures thereof. Other classes of cosmetically acceptable emollients that are liquid at 0° C., and that provide a sensory, processing, and/or property benefit may be used herein, provided that such emollients do not undesirably effect gel formation and sensory properties of the resultant composition. The amount of emollient may range from about 10 to about 80% by weight of the composition, more particularly from about 30 to about 70% by weight of the composition and, in an embodiment of particular interest, from 40 to 65% by weight of the composition.

### Deodorant Actives

Optionally, the compositions of this invention may further comprise one or more deodorant actives. Deodorant actives that may suitably employed in the subject compositions include antimicrobial agents that inhibit microbial activity by bacteriostatic or inhibitatory activity, for example, the antimicrobial agents may inhibit the metabolic activity of odor generating microbes. Deodorant actives include, but are not limited to, 2,2′-methylenebis(3,4,6,-trichlorophenol), 2,4,4′-trichloro-2′-hydroxydiphenylether (Triclosan), N-(4-chlorophenyl)-N′-(3,4-dichlorophenyl) urea (Triclocarban), 2,2′-thiobis(4,6-dichlorophenol), p-chloro-m-xylenol, dichloro-m-xylenol, polyaminopropyl biguanide, and the like. Quaternary ammonium compounds such as, for example, benzethonium chloride, cetyl pyridium chloride, cetyl pyridinium chloride, lauryl pyridium chloride, methylbenzethonium chloride, and cetyl-trimethyl ammonium bromide, and the like, may also be used in the subject compositions as antimicrobial agents. Other bacteriostats suitable for use herein are, for example, triethyl citrate, sodium N-lauroyl sarcosine, sodium N-palmitoyl sarcosine, and lauroyl sarcosine. The deodorant actives may be used alone or in combination. The amount of antimicrobial present in the compositions may range from about 0.001 to about 3% by weight of the composition, with ranges of depending upon the particular active employed. For example, when Tricolsan, available from Ciba-Geigy Corporation under the trademark, IRGASANO DP-300, is utilized, the amount thereof is typically from 0.05 to about 1% by weight of the composition, more particularly from 0.1 to about 0.5% by weight of the composition.

While the particles in the form of a synthetic oxide support on which is deposited a silver halide, also referred to herein as “silver halide clad particles” may provide an antimicrobial effect, they are considered separately from the deodorant active component(s) of the subject invention, and reference to the amounts of deodorant active present in the subject compositions does not include such particles.

### Silver Compound Clad Particles

Particles in the form of a synthetic oxide support on which is deposited a silver compound are described, for example, in U.S. Pat. No. 6,444,726 and EP 251 783, the disclosures of which are incorporated herein by reference. The synthetic oxide support of such particles preferably comprises titania (titanium dioxide), and the silver compound preferably comprises silver halide, such as, for example, silver chloride, silver bromide, and/or silver iodide, with silver chloride being of particular interest. The particles may include, as a substitute for all or a portion of the silver halide, other antimicrobial silver compounds such as, for example, silver phosophate, silver stearate, silver oxide (hydroxide), silver carbonate, silver salicylate, and the like, and mixtures thereof. Silver halides being photo- and/or thermal sensitive, it is postulated that a portion of the silver halide can be present as silver oxide and/or other species formed by reaction and/or degradation of the silver halide in the manufacture of the subject sticks. Desirably, the silver-containing compound is deposited at a loading of from about 1 to about 75% by weight of the support material, with silver compound loadings of from about 10 to about 60% of the support material being of particular interest in at least one embodiment.

In one embodiment of interest, the particle size of the support material is less than 25 microns, more particularly from about 1 to about 15 microns. The material may exist as clusters of crystallites.

As disclosed in U.S. Pat. No. 6,444,726, the silver compound clad particles may, if desired, be surface modified to provide a chemical and/or physical pathway for the silver. Surface modification may, for example, take the form of treatment with a polymer such as, for example, polyvinyl acetate, polyethylene glycol, and polyvinylpyrrolidone.

Concentrates containing the subject particles are available from Clariant AG under the trademark JM ACTICARE®.

Desirably, the silver clad particles are present in the subject compositions in an amount of from about 0.0001 to about 2% by weight. In one embodiment of interest the silver clad particles are present in the subject compositions in an amount of from about 0.001 to about 1% by weight. The amount of preference will depend, in part, on the degree of clarity desired in the gelled compositions, as amounts in excess of 0.1% can impart a somewhat hazy appearance to the stick; in certain embodiments compositions containing 0.01% by weight or less of silver clad particles may be desired. In another embodiment of interest, the gelled compositions of this invention contain from about 0.0001 to about 0.1%, more particularly from 0.001 to 0.1% by weight of such particles.

It has been found that the addition of silver halide clad particles in the subject gelled compositions may provide for a more uniform distribution of silver salt, notwithstanding that the density of the silver halide clad particles is generally greater than that of the compositions into which they are added, and that the particles are typically added while the composition is in an “un-gelled”, highly fluid state.

In one embodiment of interest the compositions of interest are free of silver dihydrogen citrate. In another embodiment of interest, at least 90%, preferably at least 95% by weight of the silver provided to the subject compositions is provided from silver halide clad particles.

### Other Components

Compositions of the invention can further comprise additional cosmetic ingredients including, but not limited to preservatives, colorants, sunscreen, chelating agents, pH adjusters, viscosity modifiers, and fragrances. In at least one embodiment of interest, the total amount of such additional optional ingredients does not exceed 8 by weight of the subject compositions. In at least one embodiment, the total amount of such additional ingredients is from 0.01 to 5% by weight, more particularly preferably from 0.1 to 3% by weight of the subject compositions.

Desirably the subject compositions are essentially free of astringent antiperspirant salts containing aluminum and/or zirconium. In at least one embodiment of interest the compositions of interest contain less than 1% by weight of antiperspirant salts containing aluminum and/or zirconium.

### Hardness Test

Preferably the gelled compositions of the subject composition have a hardness of at least 8-11 mm. As used throughout the specification and claims, the hardness of the subject composition refers to the hardness of the compositions in gelled form as measured using a penetrometer (model HPT 7138 from Precision) equipped with a 2.5 gm brass/steel tapered needle (DIN 51-579) on sticks that have been allowed to equilibrate for approximately 8-24 hours at a temperature of 26.7° C. Measurements are taken from 6 different locations on the side of sample stick, with the measurements being taken 0.5 inch (1.3 cm) apart and 0.75 inch (1.9 cm) from the sample edge), the hardness values being reported as an average of the six measurements. The depth of needle penetration into the stick is recorded to the nearest tenth of a millimeter.

### Preparation

The subject compositions can be prepared by conventional procedures. For example, the non-volatile liquid components may be combined and heated to temperatures of 70 to 90° C., the gelling agent and, if present, clarifying agent, are then introduced and the resulting blends are cooled to temperature about 2 to about 10° C. above the gelling temperature of the composition, at which time any volatile or temperature sensitive ingredients, including the silver clad particles, are introduced and the resulting composition is transferred to a conventional gel stick container at a suitable pour temperature, typically a temperature about 2 to about 5° C. above the gel temperature of the composition, and cooled to room temperature.

An embodiment of particular interest in the practice of this invention is a gelled composition which comprises:
(a) from 15 to 35% by weight water,
(b1) from 2 to 15% by weight of an alkali metal salt of a C₁₂ to C₂₄ fatty acid,
(b2) from 0.5 to 5% by weight of a polyethylene oxide-polypropylene oxide block copolymer;
(c) from 0.0001 to 1% by weight of particles in the form of a titanium dioxide support on which is deposited silver compound, preferably silver halide;
(d) from 40 to 65% by weight of emollient, wherein the wherein the emollient comprises polyhydric alcohol selected from the group consisting of propylene glycol, dipropylene glycol, butylene glycol, glycerin, sorbitol, polyethylene glycol, and mixtures thereof,
   wherein the gelled composition is in the form of a clear deodorant stick that is contained in a dispenser that includes instructions to apply the composition to the underarm, and wherein the gelled composition is essentially free of both (i) astringent antiperspirant salts containing aluminum and/or zirconium and (ii) waxy materials.

### EXAMPLES

### Example 1

Gelled sticks having the composition of Example 1 and Comparative Example A of Table 1 were prepared by blending the water, dipropylene glycol and propylene glycol components, heating the resulting blends to 85° C., adding the poloxamine to the heated blend and mixing until clear, adding the sodium stearate to the heated blends and again mixing until clear, cooling the blends to 75° C. and adding the 2-amino-2-methylpropan-1-ol with agitation, cooling the blends to 71-73° C., and mixing in the remaining ingredients. The blends were then allowed to cool to room temperature and gel.

TABLE 1

Wt. %

| | | COMPARATIVE |
|---|---|---|
| Component | EXAMPLE 1 | EXAMPLE A |
| Water | QS | QS |
| Propylene Glycol | 22.5% | 22.5% |
| Dipropylene Glycol | 40.0% | 40.0% |
| Sodium Stearate | 5.5% | 5.5% |
| Tetronic ® 1307 | 3.0% | 3.0% |
| Poloxamine | | |
| Disodium EDTA | 0.1% | 3.0% |
| 2-amino-2-methylpropan-1- | 0.4% | 0.4% |
| ol | | |
| BHT | 0.05% | 0.05% |
| Fragrance | 1.5% | 1.5% |
| Colorant | 0.005% | 0.005% |
| JM ACTICARE ® Silver | As | — |
| Chloride Suspension | 0.0002% | |
| | silver salt | |
| Silver Chloride powder | — | As 0.0002% |
| | | silver salt |

The JM ACTICARE ® Silver Chloride Suspension employed in Example 1 was an aqueous suspension of particles in the form of a titanium dioxide support on which was deposited silver chloride.

Comparative Example A exhibited settling of the silver chloride, whereas, settling of the silver-chloride containing particles was not observed in Example 1.

What is claimed is:

1. A gelled composition which comprises:
(a) from 10 to 75% by weight water,
(b) gelling agent comprising an alkali metal salt of a C₁₂ to C₂₄ fatty acid,
(c) particles in the form of a synthetic oxide support on which is deposited silver compound, and
(d) optionally, one or more emollients,
wherein the gelled composition is in the form of a clear deodorant stick.

2. A composition according to claim 1 that further includes emollient.

3. A composition according to claim 1 wherein the gelling agent further comprises a co-gellant.

4. A composition according to claim 1 wherein the silver compound comprises silver halide.

5. A composition according to claim 1 wherein the gelling agent comprises sodium stearate.

6. A composition according to claim 1 wherein the gelling agent is present in an amount of from 1 to 25% by weight of the composition.

7. A composition according to claim 3 wherein the co-gellant comprises a polyethylene oxide-polypropylene oxide block copolymer of the general structure:
R_{f}[(C₂H₄O)ₐ(C₃H₆O)_{b}(C₂H₄O)_{c}(C₃H₆O)_{d}]ₑ[H]_{g}
wherein:
R is independently selected from hydrogen, hydroxyl, C₁₀-C₂₂ fatty alkoxide, and ethylene diamine units;
a, b, c and d are independently selected integers ranging from 0 to 200 with the proviso that the sum of a, b, c and d is at least about 50;
e is an integer from 1 to 4;
f is an integer from 0 to 1; and
g is an integer from 0 to 4.

8. A composition according to claim 7 wherein the alkali metal salt of the C₁₂ to C₂₄ fatty acid is present in the composition in an amount of from 2 to 15% by weight, and the polyethylene oxide-polypropylene oxide block copolymer is present in the composition in an amount up to 6% by weight.

9. A composition according to claim 1 wherein emollient is present in an amount of from 30 to 70% by weight of the composition.

10. A composition according to claim 9 wherein the emollient comprises polyhydric alcohol selected from the group consisting of propylene glycol, dipropylene glycol, butylene glycol, glycerin, sorbitol, polyethylene glycol, and mixtures thereof.

11. A composition according to claim 1 that is essentially free of astringent antiperspirant salts containing aluminum and/or zirconium.

12. A composition according to claim 1 that is essentially free of waxy materials.

13. A composition according to claim 1 wherein water is present in the composition in an amount of 15 to 40% by weight of the composition.

14. A composition according to claim 1 wherein the synthetic oxide support comprises titanium dioxide and the silver compound comprises silver chloride.

15. A composition according to claim 14 wherein the silver halide is deposited on the synthetic oxide support a loading of from about 1 to about 75% by weight of the support.

16. A composition according to claim 1 wherein component (c) is present in the composition in an amount of from 0.001 to about 1% by weight.

17. A gelled composition which comprises:
(a) from 15 to 35% by weight water,
(b1) from 2 to 15% by weight of an alkali metal salt of a C₁₂ to C₂₄ fatty acid,
(b2) from 0.5 to 5% by weight of a polyethylene oxide-polypropylene oxide block copolymer;
(c) from 0.0001 to 1% by weight of particles in the form of a titanium dioxide oxide support on which is deposited silver halide;
(d) from 40 to 65% by weight of emollient, wherein the wherein the emollient comprises polyhydric alcohol selected from the group consisting of propylene glycol, dipropylene glycol, butylene glycol, glycerin, sorbitol, polyethylene glycol, and mixtures thereof,
wherein the gelled composition is in the form of a clear deodorant stick that is contained in a dispenser that includes instructions to apply the composition to the underarm, and wherein the gelled composition is essentially free of both (i) astringent antiperspirant salts containing aluminum and/or zirconium and (ii) waxy materials.

18. A method of inhibiting odor by the application to the human axillae of a composition according to claim 1.

19. A composition according to claim 1 that further comprises a deodorant active.

20. A composition according to claim 1 that is contained in a dispenser that includes instructions to apply the composition to the underarm.
